(19) United States
(12) Patent Application Publication
(10) Pub. No.: US 2019/0240064 A1
(43) Pub. Date: Aug. 8, 2019

(54) THUMB SUCKING CORRECTION MECHANISM

(71) Applicant: MOBIU CO., LTD., Seoul (KR)

(72) Inventor: Hyung Seok KIM, Seoul (KR)

(73) Assignee: MOBIU CO., LTD., Seoul (KR)

(21) Appl. No.: 16/329,191

(22) PCT Filed: Aug. 29, 2017
(86) PCT No.: PCT/KR2017/009402

§ 371 Date: Feb. 27, 2019

(30) Foreign Application Priority Data
Aug. 29, 2016 (KR) 10-2016-0110117

(51) Int. Cl. A61F 5/50

(52) U.S. Cl. CPC A61F 5/50

(57) ABSTRACT

The present invention relates to a thumb sucking correction mechanism wherein an opening is formed in the front surface of the upper portion of a cover body such that the thumb can bend with reference to connecting portion and can escape in the forward direction through the opening.

### TECHNICAL FIELD

The present invention relates a thumb sucking correction mechanism to prevent a finger such as the thumb from being sucked.

### BACKGROUND ART

Most children have a habit of sucking their fingers more or less. According to research, it is reported that finger sucking is a habit appearing in 40% of 1 year olds, 20% of 5 years olds and 5% of 10 years olds.

Further, finger sucking habit gradually disappears naturally over time but sometimes continues for a long time. Herein, the finger sucking habit may cause malformation of the jaw and teeth, leading to difficulties in breathing and chewing food. In severe cases, it is known to damage facial shape.

Infants may experience joy, warmth, satisfaction and comfort without tension through sucking their fingers and mostly suck their fingers when they meet various complaints, for example, they feel fear, hungry, drowsy and/or uneasiness.

Excessively blocking the finger sucking may adversely psychologically affect the infant. Therefore, careful consideration is required so that many side effects as described above due to continuous finger sucking are not caused while preventing psychological impact on infants.

Traditionally, in order to correct the finger sucking habit at home, some ways such as wrapping a finger with a bandage, applying bitter medicine to the finger, using a pacifier, covering the fingers with gloves during sleep, etc. have been used. However, these methods cause too much stress to the infant or cause a hygienic problem, thus not achieving desired effects.

In order to prevent such finger sucking habit, Korean Patent Laid-Open Publication No. 10-201.6-0057090 discloses a finger sucking corrector 10 consisting of a finger cap 20, a wristband 30 placed at a distance to the finger cap 20, a joint 40 for connecting the finger cap 20 and the wristband 30, and a finger ring 50 mounted on one side of the finger cap 20, as shown in FIG. 1, which are integrally formed.

The finger sucking corrector 10 is made of a material selected from silicone or synthetic resin harmless to the human body and comprises the finger cap 20, the wristband 30, the joint 40 and the finger ring 50 integrally formed together, thereby having benefits such as ease of storage and cleaning.

The finger cap 20 is formed in a hollow shape such that a finger of the infant can be inserted into an inner space 21, and has an air passage 22 formed around an inner periphery thereof for air circulation. Further, the top 23 of the finger cap 20 is formed to be curved or inclined inward while the bottom 24 thereof is formed to be curved towards the outside. That is, the finger cap 20 has the top portion 23 formed to be curved inward such that the infant can put the same in the mouth without difficulty and the bottom portion 24 formed to be curved such that a finger of the infant can be easily inserted.

However, since the conventional finger cap surrounds the outer periphery of first and last knuckles of the thumb in order to prevent finger sucking, the infant cannot bend the last knuckle toward the first knuckle to escape the finger cap to thus cause difficulty in grasping objects and may be apt to feel discomfort. Accordingly, there are problems such that activity of fingers of a child is lowered and touch recognition with fingers is decreased.

### PRIOR DOCUMENT

### Patent Document

Korean Patent Laid-Open Publication No. 10-2016-0057090

### DISCLOSURE

### Technical Problem

The present invention has been devised to solve the aforementioned problems and an object of the present invention is to provide a thumb sucking correction mechanism that may reduce thumb sucking to correct a finger sucking habit and enable easier bending behavior of the thumb at the same time.

### Technical Solution

In order to achieve the above object, the present invention provides a thumb sucking correction mechanism, which includes: a cover body with an open bottom wherein a thumb is inserted into and wrapped by the same; and a joint member connected to opposite sides of a front portion of the cover body at opposite ends of the joint member, wherein an upper portion of the cover body has a closed part including closed top surface and closed rear surface of the cover body and an opening formed on an open front surface of the cover body, and wherein the thumb may be bent with reference to the joint member and may escape in a front direction through the opening.

Further, the joint member may be provided in plural and spaced apart in the vertical direction.

Further, the thumb sucking correction mechanism may further include a first band and a second band provided on a lower portion of the cover body to extend forward, wherein one end of the first band is inserted into an insertion port formed in the second band and thus coupled to each other.

Further, the thumb sucking correction mechanism may further include a protrusion protruding from opposite sides in the cover body wherein the protrusion is formed to extend up to inner surfaces of the first band and the second band.

Further, the thumb sucking correction mechanism may further include a lateral protrusion protruding from opposite sides of an outer surface of the cover body, or a rear protrusion protruding rearward from an outer rear surface of the cover body, wherein the lateral protrusion and the rear protrusion are formed to protrude outward with going to the lower portion.

Further, the thumb sucking correction mechanism may further include an auxiliary cover body configured to wrap a index finger or a middle finger, wherein a third band and a fourth band extending from a lower portion of the auxiliary cover body have ring members extending from the bands, respectively, and wherein the first band and the second band are inserted through the ring members and coupled to the third band and the fourth band, respectively.

Further, the thumb sucking correction mechanism may further include a plurality of inner protrusions protruding from an inner rear surface of the cover body, which extend in the vertical direction.

### Advantageous Effects

According to the present invention, the following advantages may be obtained.

Thumb sucking may be reduced to thus correct a thumb sucking habit. At the same time, the thumb can be bent to escape in the front direction of the cover body, so that an infant may easily move the thumb and grip objects. Accordingly, it is possible to prevent degradation of touch recognition by the thumb.

Further, the joint member is provided in plural and spaced apart in the vertical direction so as to prevent escape of the thumb in a larger range of vertical width while covering less area of the inner side of the thumb.

Further, one end of the first band is inserted into the insertion port of the second band and coupled to the second band, thereby easily fixing the cover body in a desired position.

Further, a portion at which the protrusion is formed has a larger thickness and a higher tensile strength, compared to the other portion of the cover body, so as to restrain easy stretching of the above portion, thereby preventing the thumb from escaping the cover body. Further, the product may be effectively prevented from loosening due to repeated use. Further, when an internal protrusion protruding inside the cover body touches the back or palm of a hand (in case of line contact) of an infant, the other portions of the cover body, the first band and the second band may be in relatively little contact with the back or palm of the hand of the infant, thereby allowing smooth fresh air inlet from the outside and achieving an advantage of better air ventilation.

Further, the lateral protrusion and the rear protrusion are effective to avoid the cover body undesirably stretching, and may be sucked instead of the thumb to thus have effects of replacing the thumb.

Further, the first band may be coupled to the third band while coupling the second band to the fourth band, and therefore, the auxiliary cover body configured to wrap the index finger and the middle finger and the main cover body can be compatible with each other and easily used.

Further, owing to the internal protrusion inside the cover body, the other portion of the cover body may be in relatively little contact with the thumb of the infant to thus allow smooth fresh air inlet from the outside and achieve an advantage of better air ventilation.

### DESCRIPTION OF DRAWINGS

FIG. 1 is a perspective view illustrating a conventional finger sucking correction mechanism.

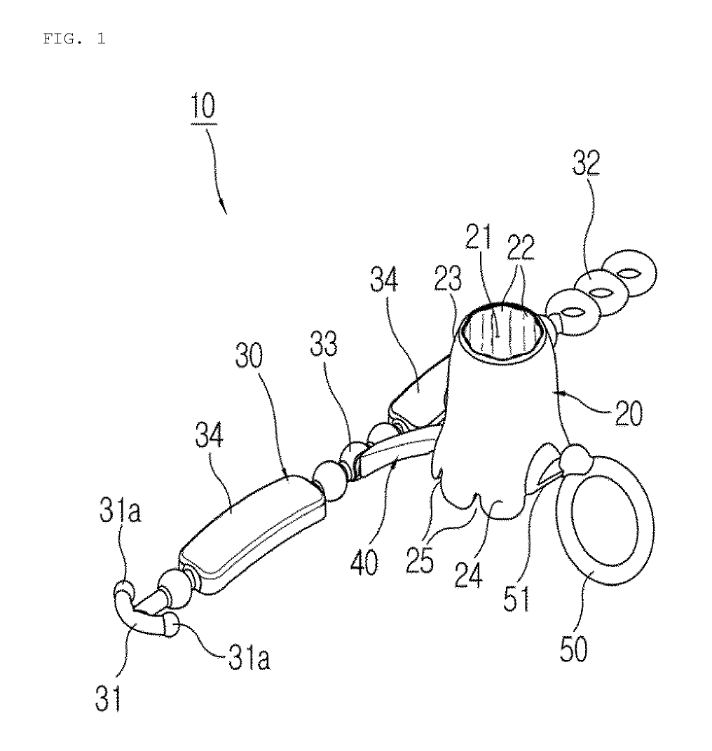

FIG. 2 is a perspective view illustrating a thumb sucking correction mechanism according to a first preferable embodiment of the present invention (without an auxiliary cover body).

FIG. 3 is a side directional view of FIG. 2.

FIG. 4 is a perspective view illustrating a first band and a second band coupled to each other.

FIG. 5 is a perspective view illustrating the thumb sucking correction mechanism in a state of being worn on a hand.

FIG. 6 is a perspective view illustrating an auxiliary cover body.

FIG. 7 is a rear side view illustrating the cover body and the auxiliary cover body coupled to each other.

FIG. 8 is a perspective view illustrating a thumb sucking correction mechanism according to a second embodiment of the present invention (without an auxiliary cover body).

FIG. 9 is a side directional view of FIG. 8.

FIG. 10 is a perspective view illustrating a thumb sucking correction mechanism according to a third embodiment of the present invention (without an auxiliary cover body).

### BEST MODE

Hereinafter, the present invention will be described in detail with reference to the accompanying drawings.

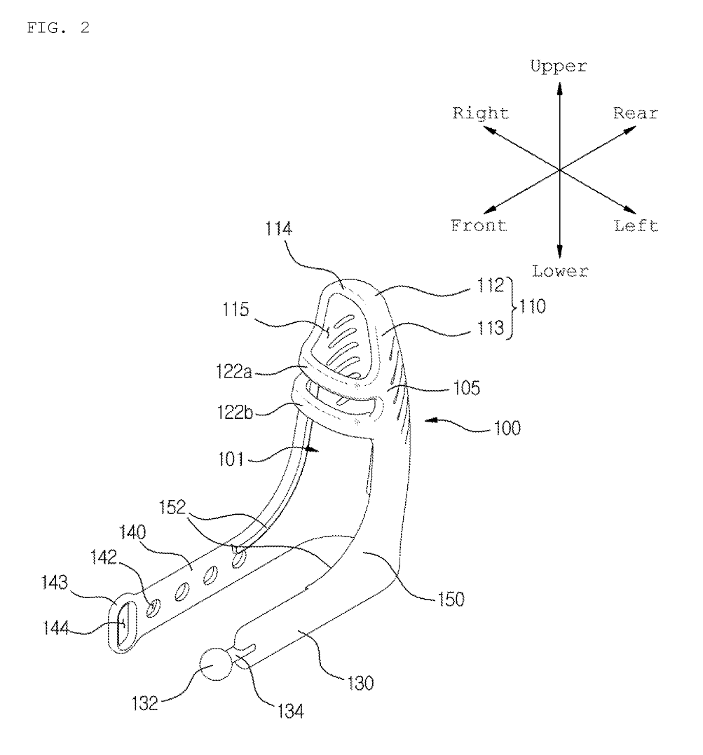

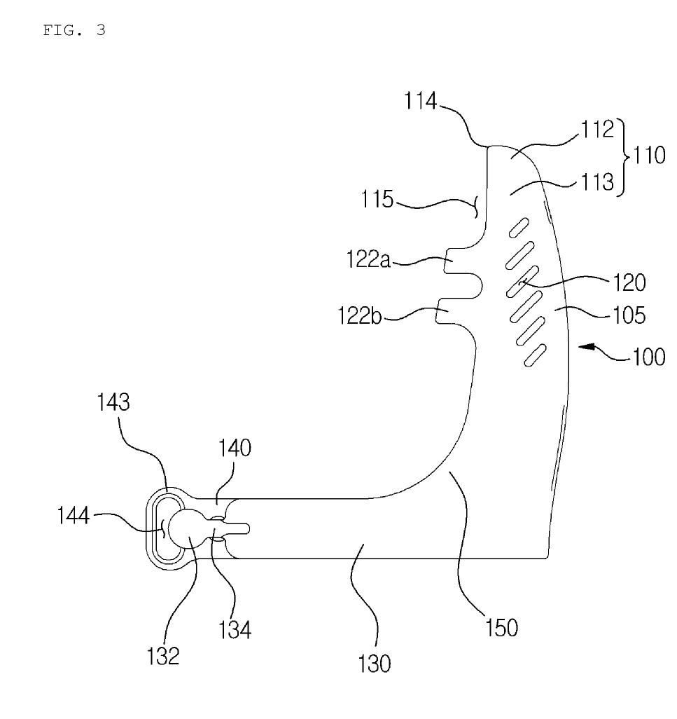

As shown in FIGS. 2 to 5, the thumb sucking correction mechanism according to the first preferable embodiment of the present invention includes: a cover body 100 with an open bottom wherein a thumb 200 is inserted into and wrapped by the same; and joint members 122a and 122b connected to opposite sides of a front portion of the cover body 100, wherein an upper portion of the cover body 100 has a closed part 110 including closed top surface 112 and closed rear surface 113 of the cover body and an opening 115 formed on an open front surface of the cover body, and wherein the thumb 200 is bent with reference to the joint member 122a and may escape in the front direction through the opening 115.

Further, the thumb sucking correction mechanism further includes a first band 130 and a second band 140 provided at opposite sides of a lower portion of the cover body 100 to extend forward.

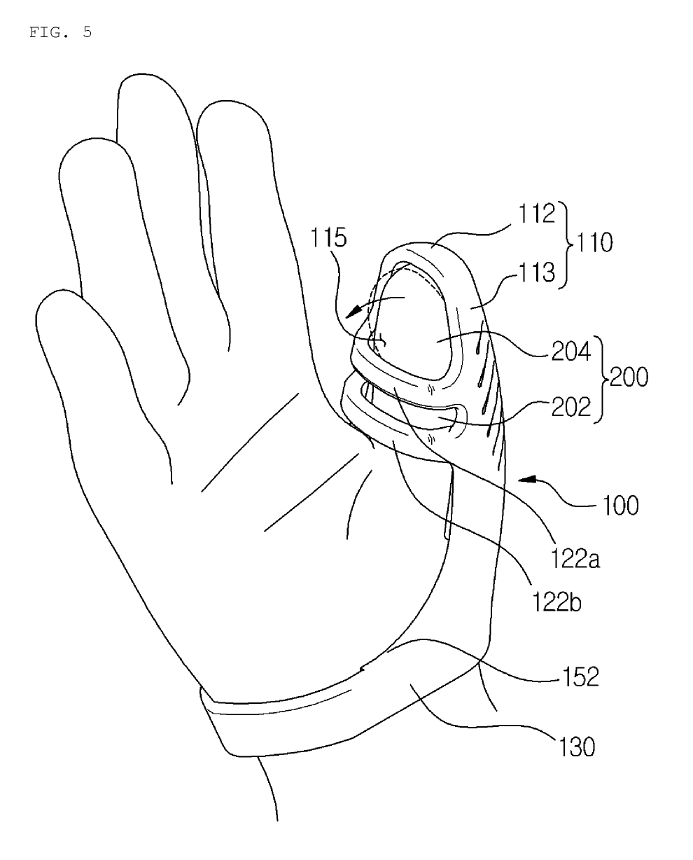

As shown in FIGS. 2 to 5, the cover body 100 has a lower portion and a front portion 101 which are open, wherein a horizontal cross-section is formed in a front-open semicircular shape and extends in the vertical direction. Therefore, a space into which a thumb 200 is inserted may be provided in the cover body 100 and, as shown in FIG. 5, the thumb 200, the palm of a hand and a part of the back of the hand may be put in and wrapped by the cover body. The cover body 100 may be made of a silicone material to thus be flexible.

Meanwhile, an upper portion of the cover body 100 is provided with a closed part 110 including closed top surface 112 and closed rear surface 113 of the cover body and an opening 115 formed on an open front surface of the cover body.

The rear surface 113 on the upper portion of the cover body 100 may be formed as a rearward-convex curved surface while having a horizontal cross-section formed in a front-open semicircular shape. The rear surface 113 on the upper portion of the cover body 100 is connected to the top end of a rear surface 105 in the center of the cover body 100 and extends upward. An inner width of the rear surface 113 on the upper portion of the cover body 100 decreases with going to the upper portion. As shown in FIG. 5, the last knuckle 204 of the thumb 200 with a finger nail may be inserted into and placed inside the rear surface 113 on the upper portion of the cover body 100. Further, the rear surface 113 on the upper portion of the cover body 100 may cover the outside of the last knuckle 204 of the thumb 200. An inner width of the upper portion of the cover body 100 is formed smaller than the inner width of the center of the cover body 100.

Further, a top surface 112 formed in a curved shape is placed at the top end of the rear surface 113 on the upper portion of the cover body 100. In other words, a bottom end of the top surface 112 on the upper portion of the cover body 100 may be connected to the top end of the rear surface 113 on the upper portion of the cover body 100. Further, the top surface 112 on the upper portion of the cover body 100 has an inflection point (or a turning point) 114 in the front of the top end. The top surface 112 on the upper portion of the cover body 100 may be formed as a curved surface which is inclined downward in left, right and rear directions. As shown in FIG. 5, an upper portion of the last knuckle 204 of the thumb 200 inserted into the cover body 100 may be covered and closed by the top surface 112 on the upper portion of the cover body 100.

Meanwhile, the front of the closed part 110, that is, a front surface of the upper portion of the cover body 100 is open to form an opening 115. The opening 115 is formed throughout the front of the rear surface 113 and the front of the top surface 112 on the upper portion of the cover body 100. Accordingly, the last knuckle 204 of the thumb 200 inserted into the closed part 110 may be bent forward relative to the first knuckle 202, thereby protruding in the front direction of the cover body 100 through the opening 115. In other words, the last knuckle 204 of the thumb 200 can escape in the front direction of the cover body 100 through the opening 115.

Further, a plurality of thru-holes 120 is formed by perforating the cover body 100 in order to allow communication between the inside and the outside of the cover body. The thru-hole 120 may be formed in a long hole shape.

Further, opposite ends of the joint members 122a and 122b are connected at opposite sides in the front of the cover body 100. Each of the joint members 122a and 122b may be formed in a forward-convex curved band shape and then connected to opposite sides in the front of the center of the cover body 100. In other words, opposite ends of the joint members 122a and 122b are connected at opposite sides in the front of the rear surface 105 in the center of the cover body 100. The joint members 122a and 122b are formed to be convex in the front direction while having a horizontal cross-section formed in a rear-open semicircular shape.

The joint members 122a and 122b may serve to cover a part of the open front portion 101 of the cover body 100. Therefore, as shown in FIG. 5, when the thumb 200 is inserted into the cover body 100, an inner side of the first knuckle 202 of the thumb 200 can be covered.

Further, the joint members 122a and 122b may be provided in plural and spaced apart in the vertical direction. The joint members 122a and 122b may include a first joint member 122a formed at the upper side and a second joint member 122b formed at the lower side thereof. Therefore, it is possible to prevent escape of the thumb 200 in a larger range of vertical width while covering less area of the inner side of the thumb 200.

As shown in FIG. 5, when the thumb 200 of an infant is inserted into the cover body 100, the last knuckle 204 of the thumb 200 is placed on the upper portion of the cover body 100, that is, inside the closed part 110, while the first knuckle 202 of the thumb 200 is placed in the center of the cover body 100. To explain again, the last knuckle 204 of the thumb 200 is positioned inside the opening 115 while the first knuckle 202 of the thumb 200 is positioned inside the joint members 122a and 122b. Accordingly, the thumb 200 of the infant may be bent with reference to the joint members 122a and 122b, more particularly, the first joint member 122a, and can escape in the front direction through the opening 115. Since the last knuckle 204 of the thumb 200 is bent forward relative to the first knuckle 202, the thumb 200 can be bent with reference to the first joint member 122a.

As described above, it is possible to cover the outside and the top side of the thumb 200, thereby reducing thumb sucking 200 and correcting a thumb sucking habit. At the same time, it is possible to bend the thumb 200 so as to escape the cover body 100 in the front direction thereof, thereby ensuring easy or convenient movement of the thumb 200. Therefore, the infant may feel less discomfort and can easily perform a motion such as grasping an object, while preventing degradation of touch recognition by the thumb 200.

The conventional correction devices prevented bending of the thumb 200 in order to pull out the last knuckle 204. In other words, since the conventional correction device surrounds peripheries of the first knuckle 202 and the last knuckle 204 of the thumb 200, the thumb 200 could not escape to the outside of the correction device while bending the last knuckle 204 of the thumb 200 relative to the first knuckle 202 of the same, hence causing difficulty in grasping objects. Further, it was apt to feel uncomfortable. As a result, the conventional correction device disturbed movement of the thumb 200 of the infant and had a problem of degrading touch recognition through fingers.

On the other hand, a first band 130 and a second band 140 are provided to extend from opposite sides in the front of the lower portion of the cover body 100. Further, a slope 150 is present on each upper portion of the first band 130 and the second band 140, respectively, wherein the slope has a top surface inclined upward with going to the rear side.

Meanwhile, a protrusion 152 linearly protruding from opposite sides in the cover body 100 and extending to inner sides of the first band 130 and the second band 140 is provided. The protrusion 152 is bent in a curved shape along an inner surface of the slope and extends inward. The protrusion 152 extends to upper inner sides of the first band 130 and the second band 140 in view of the front direction, while the protrusion 152 extends along opposite inner front ends of the cover body 100 and up to the lower portion of the second joint member 122b. A portion of the cover body 100 made of a silicone material, at which the protrusion 152 is formed, has a larger thickness and a higher tensile strength, compared to the other portion, so as to restrain easy stretching of the above portion. Therefore, it is possible to prevent the thumb 200 inserted into the upper portion of the cover body 100 from easily escaping to the lower portion. Further, a product may be effectively protected not to loosen due to repeated use.

Further, when the thumb sucking correction mechanism is worn on a thumb of an infant and the protrusion protruding inward touches the back or palm of a hand (in case of line contact) of the infant, the other portions of the cover body 100, the first band 130 and the second band 140 may be in relatively little contact with the back or palm of the hand of the infant, thereby allowing smooth fresh air inlet from the outside and achieving an advantage of better air ventilation.

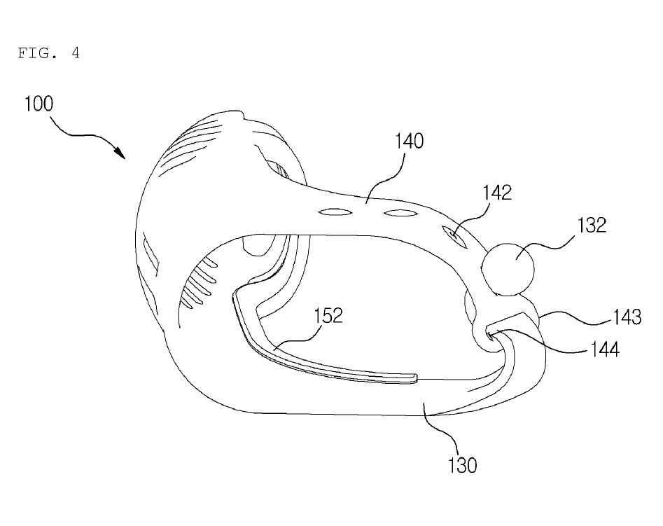

Further, the first band 130 and the second band 140 may be coupled to each other by inserting one end of the first band 130 into an insertion port 142 formed in the second band 140 (see FIGS. 2 and 4).

In more detail, the first band 130 is formed at one side in the front of the lower portion of the cover body 100 to extend forward. Further, an extension 134 extending forward in the center of one end of the first band 130 and a coupling member 132 protruding in a spherical shape at one end of the extension 134 are provided. Up and down and right and left widths of the coupling member 132 are larger than those of the extension 134. The coupling member 132 may be forcibly inserted into the insertion port 142 of the second band 140.

Further, the second band 140 is provided with a coupling ring 143 at one end thereof. A thru-hole 144 through which the one end of the first band 130, that is, the coupling member 132 can pass, is formed inside the coupling ring 143. Further, the second band 140 has the insertion port 142 into which the coupling member 142 is forcibly inserted and fixed. The insertion port 142 may be provided in plural in a length direction of the second band 140 and spaced apart at a predetermined interval.

As shown in FIG. 4, after the coupling member 132 passes through a hole 144 of the coupling ring 143 from the outside of the second band 140 to the inside thereof, the first band 130 is forcibly inserted to pass through the insertion port 142 from the inside of the second band 140 to the outside thereof, thereby coupling the first band and the second band to each other. As a result of the coupling described above, the infant cannot easily release the coupling of the first band 130 and the second band 140.

A width (diameter) of the coupling member 132 formed in a spherical shape is larger than an inner width of the insertion port 142 so that the coupling member may push and broaden the insertion port 142 in order to increase the inner width of the insertion port, thereby being forcibly inserted therein. Depending upon a location of the insertion port 142 into which the coupling member 132 is inserted, lengths of the first band 130 and the second band 140 coupled to each other may be adjustable. The second band 140 may be formed longer than the first band 130.

As shown in FIG. 5, a part of the hand below the thumb 200 may be positioned inside the lower portion of the cover body 100. The first band 130 may surround the lower portion of the palm of the hand or the wrist, while the second band 140 may surround the back of the hand or an outer side of the wrist. Thereafter, opposite of the first band and the second band may be coupled to each other. Through the coupling of the first band 130 and the second band 140, the cover body 100 may be easily fixed in a desired position while capping the thumb 200.

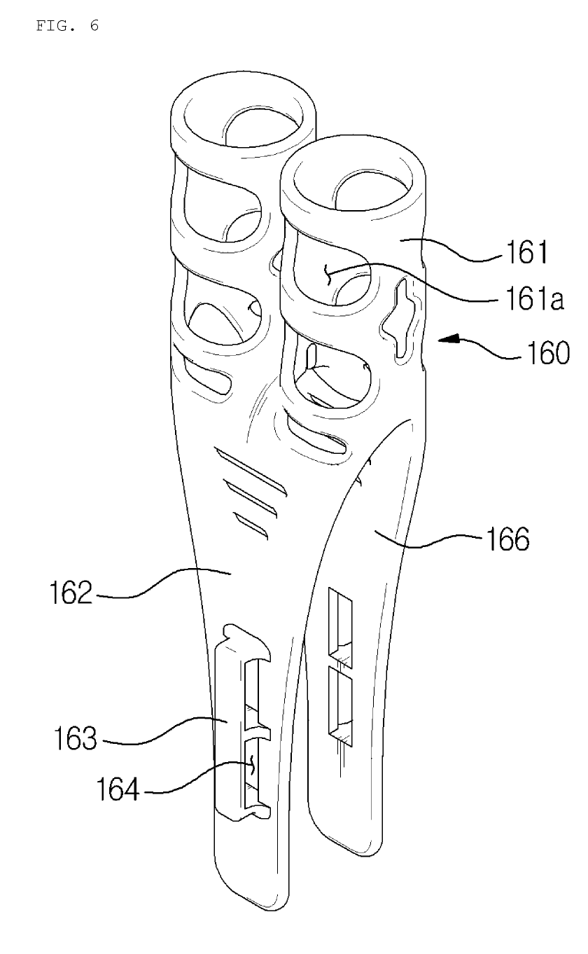

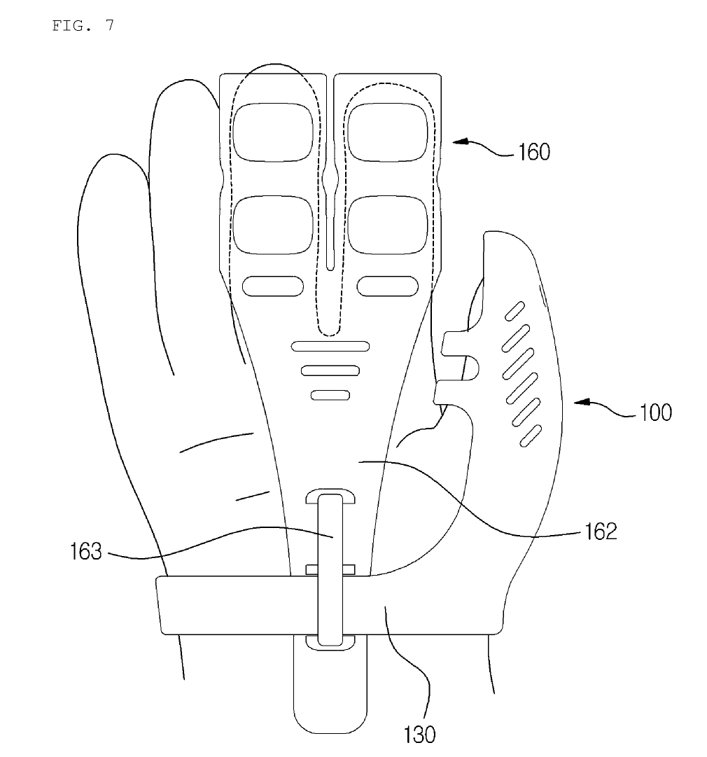

Meanwhile, as shown in FIGS. 6 and 7, the thumb sucking correction mechanism further includes an auxiliary cover body 160 configured to wrap the index finger or the middle finger. The auxiliary cover body 160 includes two hollow cylindrical members 161 formed to wrap the index finger and the middle finger inserted therein. In the front and rear sides of each hollow cylindrical member, openings 161a are formed in plural, respectively.

A third band 162 and a fourth band 166 are provided at a lower portion of the auxiliary cover body to extend downward. Protrusion type ring members 163 are formed at the outside of the third band 162 and the fourth band 166, respectively. There are spaces 164 inside the ring members 163 into which the first band 130 and the second band 140 are inserted through the ring members. Each ring member 163 may include the space 164 partitioned in the vertical direction and formed into a plurality of compartments.

As shown in FIG. 7, the first band 130 may pass through the space 164 (see FIG. 6) inside the ring member 163 and then be coupled to the third band 162. Similarly, the second band 140 may be coupled to the fourth band 166. Therefore, the auxiliary cover body 160 configured to wrap the index finger or the middle finger and the (main) cover body 100 may be compatible with each other and easily used.

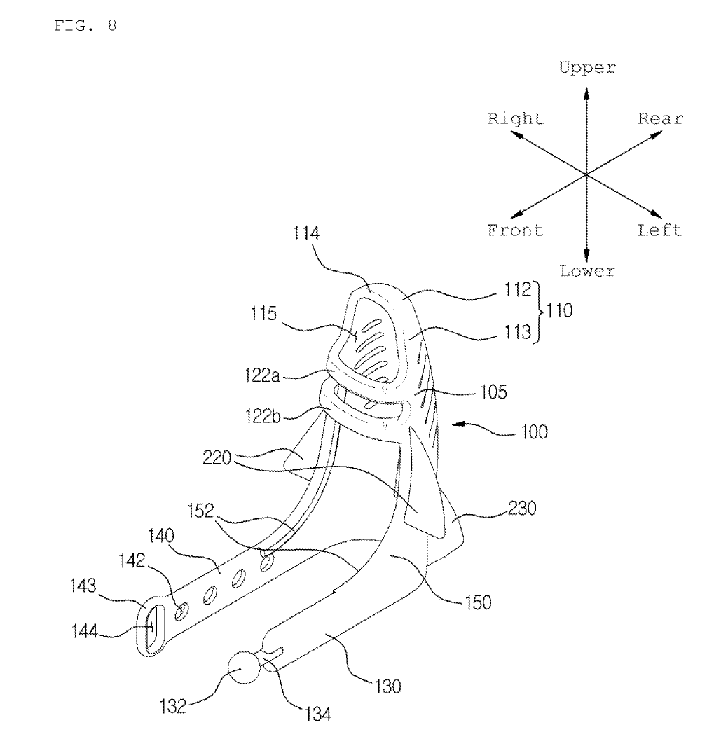

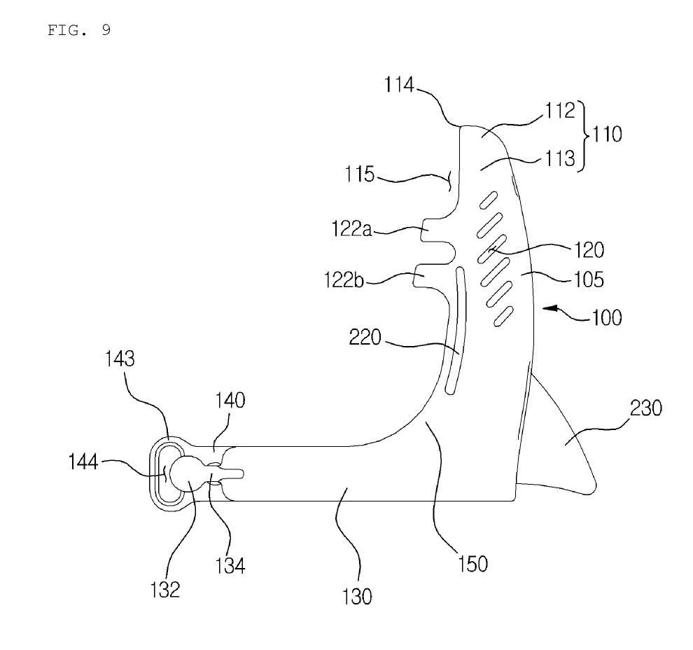

Alternatively, FIGS. 8 and 9 illustrate a thumb sucking correction mechanism according to a second embodiment of the present invention. The thumb sucking correction mechanism shown in FIGS. 8 and 9 further includes lateral protrusions 220 protruding from opposite sides of an outer surface of the cover body 100 and a rear protrusion 230 protruding rearward from an outer rear surface of the cover boy 100. The lateral protrusions 220 are formed on opposite sides of the outer surface of the cover boy 100, wherein an upper portion of each lateral protrusion 220 is formed at a point contacting with opposite ends of the second joint member 122b. Further, the rear protrusion 230 is formed on a lower portion of the outer rear surface of the cover body 100.

Each of the lateral protrusions 220 and the rear protrusion 230 may be formed in a shark's fin-like triangular shape (generally called a sawtooth shape), and protrude outward with going to the lower portion. The lateral protrusions 220 and the rear protrusion 230 effectively prevent the cover body from undesirably stretching, and may be sucked instead of the thumb 200 to thus have effects of replacing the thumb.

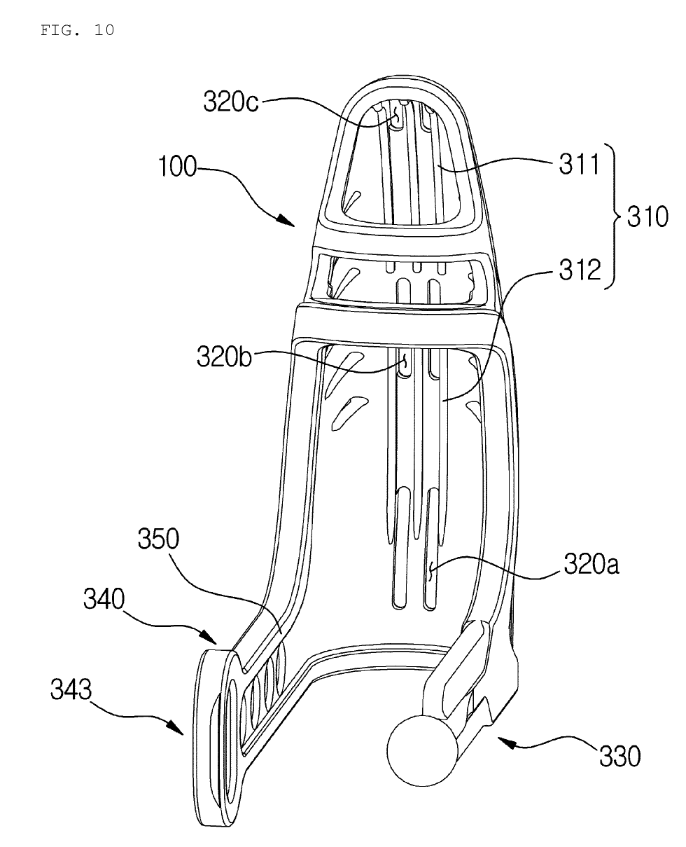

Alternatively, FIG. 10 illustrates a thumb sucking correction mechanism according to a third embodiment of the present invention.

The same configurations as the first embodiment could be understood with reference to the above description of the first embodiment, while the following description will be given only for specific configurations of the third embodiment.

As shown in FIG. 10, a plurality of inner protrusions 310 formed at the inner rear surface of the cover body 100 to protrude and extend in the vertical direction is included. The inner protrusions 310 are formed in the vertical direction while substantially protruding in the front direction.

More particularly, the inner protrusions 310 include a first inner protrusion 311 formed on an upper portion of the inner rear surface of the cover body 100, and a second inner protrusion 312 extending from the center to the lower portion of the cover body. The first inner protrusion 311 and the second inner protrusion 312 may be spaced apart in the vertical direction.

The first inner protrusion 311 may be provided in plural, especially, three (3) first inner protrusions 311 are spaced apart from one another in right and left direction, that is, the lateral direction. Further, two (2) first rear thru-holes 320c are formed between the spaced 3 first inner protrusions 311. The first rear thru-hole 320c is formed by perforating the rear surface of the cover body 100 in order to allow communication between the inside and the outside of the cover body 100.

Likewise, three (3) second inner protrusions 312 are also formed and spaced apart from one another in the lateral direction. Further, two (2) second rear thru-holes 320b are formed on an upper portion between the spaced 3 second inner protrusions 312 while two (2) third rear thru-holes 320a are formed on a lower portion between the above protrusions 312. The second rear thru-holes 320b and third rear thru-holes 320a are formed by perforating the rear surface of the cover body 100 in order to allow communication between the inside and the outside of the cover body 100, wherein each of these thru-holes may be formed in a long hole shape.

When the thumb sucking correction mechanism is worn on a thumb of a user (that is, an infant) and the thumb 200 is inserted into the cover body 100, the inner protrusions 310 of the cover body 100, more particularly, the first inner protrusion 311 and the second inner protrusion 312 may contact an outer portion of the thumb (in particular, a portion with a thumbnail) and thus the other portion of the cover body 100 may be in relatively little contact with the thumb of the infant, thereby allowing smooth fresh air inlet from the outside and achieving an advantage of better air ventilation through the first rear thru-hole 320c, the second rear thru-hole 320b and the third rear thru-hole 320a.

Further, as shown in FIG. 10, a stepped part 350 is formed at the top end and the bottom end of a second band 320, wherein the stepped part protrudes inward and outward to thus have a larger thickness, compared to the center portion of the second band. The stepped part 350 extends in a length direction of the second band 340. Further, a thickness of a coupling ring 343 connected to the end of the second band 340 is substantially close to the thickness of the stepped part 350 rather than the center portion of the second band 340.

Further, a front stepped part (numeral not given) is formed at opposite ends of the cover body 10, wherein the stepped part protrudes inward and outward from the bottom to the center portion and is stepped. Similarly, a bottom stepped part (numeral not given) is formed at the bottom end of the cover body 100, wherein the stepped part protrudes inward and outward and is stepped. Further, a portion of a first band 330 is stepped inward and outward relative to the other portion thereof and has a larger thickness, compared to the other portion.

As described above, the stepped part 350, the front stepped part, the bottom stepped part and the stepped portion of the first band may have a larger thickness and a higher tensile strength, compared to the other portion of the cover body 100, so as to restrain easy stretching of the above portions, thereby preventing the thumb inserted in the cover body 100 from escaping to the lower portion. Further, a product may be effectively protected not to be loosened due to repeated use.

As such, although the present invention has been described with reference to preferred embodiments of the preset invention, one having ordinary knowledge and skill in the art will implement various modifications or variations in an extent not departing from the spirit and scope of the invention defined in appended claims below.

### DESCRIPTION OF SYMBOLS

100: Cover body
101: Front portion
105: Rear surface (center portion of cover body)
110: Closed part
112: Top surface (upper portion of cover body)
113: Rear surface (upper portion of cover body)
114: Inflection point (turning point)
115: Opening
120: Thru-hole
122a: First joint member
122b: Second joint member
130, 330: First band
132: Coupling member
140, 340: Second band
142: Insertion port
143, 343: Coupling ring
144: Hole
150: Slope
152: Protrusion
160: Auxiliary cover body
161: Cylindrical portion
161a: Opening
162: Third band
163: Ring member
164: Space
166: Fourth band
200: Thumb
202: First knuckle
204: Last knuckle
220: Lateral protrusion
230: Rear protrusion
310: Inner protrusion
311: First inner protrusion
312: Second inner protrusion
320a: Third rear thru-hole
320b: Second rear thru-hole
320c: First rear thru-hole
350: Stepped part

1. A thumb sucking correction mechanism, comprising:
a cover body with an open bottom wherein a thumb is inserted into and wrapped by the same; and
a joint member connected to opposite sides of a front portion of the cover body at opposite ends of the joint member,
wherein an upper portion of the cover body has a closed part including closed top surface and closed rear surface of the cover body and an opening formed on an open front surface of the cover body, and
wherein the thumb is bent with reference to the joint member and can escape in a front direction through the opening.

2. The correction mechanism according to claim 1, wherein the joint member is formed in plural and spaced apart from each other.

3. The correction mechanism according to claim 1, further comprising a first band and a second band formed on a lower portion of the cover body to extend forward,
wherein one end of the first band is inserted into an insertion port formed in the second band and thus coupled to each other.

4. The correction mechanism according to claim 3, further comprising a protrusion protruding from opposite sides in the cover body, wherein the protrusion is formed to extend up to inner surfaces of the first band and the second band.

5. The correction mechanism according to claim 3, further comprising lateral protrusions protruding from opposite sides of an outer surface of the cover body or a rear protrusion protruding outward from an outer rear surface of the cover body,
wherein the lateral protrusions and the rear protrusion are formed to protrude outward with going to the lower portion.

6. The correction mechanism according to claim 3, further comprising an auxiliary correction body formed to wrap a index finger or a middle finger,
wherein a third band and a fourth band extending from a lower portion of the auxiliary cover body have protrusion type ring members, respectively,
wherein the first band and the second band are inserted through the ring members and thus coupled to the third band and the fourth band, respectively.

7. The correction mechanism according to claim 3, further comprising a plurality of inner protrusions, which protrudes from an inner rear surface of the cover body and extends in the vertical direction.
